(19) United States
(12) Patent Application Publication
(10) Pub. No.: US 2010/0129457 A1
(43) Pub. Date: May 27, 2010

(54) Nanodiamond Enhanced Drugs

(76) Inventor: Ali Razavi, Dallas, PA (US)

Correspondence Address: ZALE Patent Law, 121 Old Orchard Road, Clarks Summit, PA, 18503-2053, US

(21) Appl. No.: 12/626,614

(22) Filed: Nov. 25, 2009

Related U.S. Application Data
(60) Provisional application No. 61/118,281, filed on Nov. 26, 2008.

(51) Int. Cl. A61K 9/14

(52) U.S. Cl. 424/489; 977/906; 977/773

(57) ABSTRACT

Drugs 11 and other functional groups are attached to surfaces of nano-sized diamonds (NDs) 20 to enhance the efficacy of drugs 11 such as analgesics, cholesterol-reducing drugs and other substances. Such coatings are formed by covalently linking NDs to the drug 11. NDs 20, due to its small size and spherical shape exhibits a large surface area which enhances contact with other substances and therefore the chemical reactions. The large surface area of NDs 20 covered with active drugs allows greater access to active sites of the drugs and allows a large amount of substance to come in contact with other chemical entities enhancing the reactions. NDs 20 are also a solution stabilizer allowing enhanced concentration and solubility of coated NDs 20, enhancing the ability of the active sites of drugs 11 to be dissolved and come in contact with corresponding sites 13,15 of other entities enhancing their efficacy.

### CROSS REFERENCE TO RELATED APPLICATIONS

This patent application is a U.S. Continuation-In-Part Utility Patent Application claiming priority from U.S. Provisional Patent Application “Nanodiamond Enhanced Drugs” Ser. No. 61/118,281, filed Nov. 26, 2008.

This application is also related to U.S. Utility Patent Application “Nanodiamond Enhanced Efficacy” Ser. No. 12/399,844 filed Mar. 6, 2009 which was from U.S. Provisional Patent Application “Nanodiamond Enhanced Efficacy” U.S. Ser. No. 61/034,173 filed Mar. 6, 2008.

The present application is also related to U.S. Utility Patent Application “Multifunctional Articles And Method For Making The Same”, Ser. No. 12/301,356 filed Nov. 18, 2008 that was from PCT patent application “Multifunctional Articles and Method for Making The Same” Appl. No. PCT/US2007/016,194 filed Jul. 17, 2007 that was from U.S. Provisional Patent Application “Biofunctional Articles For Personal Care Applications and Method of Making the Same” Ser. No. 60/831,438, filed Jul. 18, 2006.

### BACKGROUND OF THE INVENTION

1. Field of the Invention

The present invention relates to a substance and method of enhancing the efficacy of drugs and more specifically a substance and method of increasing the efficacy of drugs such as analgesics and cholesterol inhibiting drugs.

2. Discussion of Related Art

There has always been a need to increase the efficacy of various drugs and preparations. One such useful class of drugs is the analgesic class. Analgesics are used to reduce pain. Analgesics are effective when used in the proper level but lose their ability as the concentration drops below a critical concentration. This may be due to the fact that enough active sites on drug must make contact with pain receptors or intermediate chemicals involved in nerve transmission.

Another class of drugs which is important is the cholesterol reducing class of drugs. The active sites of the cholesterol reducing drugs must come in contact with cholesterol or its precursors to be effective. Again, when the concentrations become low, the effect is reduced. This is true of most drugs in general.

Drugs are typically used in an aqueous solution inside of a person or animal. Molecules flowing in a solution are randomly dispersed and oriented. Also, since drugs flow in solution to attach to other entities, it is important to have a large amount of the drugs in solution, increasing the local concentration and the number of drug molecules attaching reacting. The higher concentration causes higher costs and other problems.

Currently, there is a need for drugs which are more soluble in a fluid, and are more effective for a given concentration.

### SUMMARY OF THE INVENTION

One embodiment of the present invention is a method of enhancing efficacy of a drug comprising the steps of:
a) acquiring a plurality of diamond particles having a plurality of molecules on its surface, the diamond particles having a diameter of less than 10 nanometers;
b) covalently attaching a plurality of intermediate entities to the molecules on the surface of the diamond particles, and
c) replacing at least a portion of the attached intermediate entities attached to the surface of the diamond particles with drug molecules positioned in an orderly array with their active sites facing substantially outward to create coated diamond particles exhibiting enhanced drug efficacy.

Another embodiment of the present invention is the method described above wherein the drug is an analgesic drug.

Another embodiment of the present invention is the method described above wherein the drug is a cholesterol-reducing drug.

Another embodiment of the present invention is the method described above wherein the nanodiamond-drug complex is used as a cholesterol-reducing drug.

### OBJECTS OF THE INVENTION

It is an object of the present invention to enhance the potency of a conventional drug.

It is another object of the present invention to enhance the solubility of conventional drugs.

It is another object of the present invention to provide a method of amplifying the effect of a drug in-situ.

It is another object of the present invention to provide a method of holding drug molecules in an orientation to maximize their reactivity.

It is another object of the present invention to provide a method for locally increasing the effective concentration of a drug while keeping the overall concentration constant.

It is another object of the present invention to an analgesic drug exhibiting enhanced efficacy.

It is another object of the present invention to provide a cholesterol-reducing drug exhibiting enhanced efficacy.

### BRIEF DESCRIPTION OF THE DRAWINGS

The advantages of the instant disclosure will become more apparent when read with the specification and the drawings, wherein:

FIG. 1 is a schematic illustration of how molecules react under normal prior art conditions.

FIG. 2 is a schematic microscopic view of a portion of a nano-diamond showing the structure of chemical entities attached to the surface of the nano-diamond.

FIG. 3 is an illustration of a chemical reaction for coating nano-diamonds with an intermediary according to one embodiment of the present invention.

FIG. 4 is an illustration of a chemical reaction for coating nano-diamonds with an intermediary according to another embodiment of the present invention.

FIG. 5 is an illustration of a chemical reaction for coating nano-diamonds with an intermediary according to another embodiment of the present invention.

FIG. 6 is an illustration of a chemical reaction for substituting an intermediaries covering nanodiamond's surfaces with a drug according to the present invention.

### DETAILED DESCRIPTION OF THE INVENTION

### Definitions

In describing and claiming the present invention, the following terminology will be used in accordance with the definitions set forth below.

The singular forms “a,” “an,” and “the” include plural referents unless the context clearly dictates otherwise. Thus, for example, reference to “a solvent” includes reference to one or more of such solvents, and reference to “the dispersant” includes reference to one or more of such dispersants.

As used herein, “formulation” and “composition” may be used interchangeably and refer to a combination of elements that is presented together for a given purpose. Such terms are well known to those of ordinary skill in the art.

As used herein, “biological material” refers to any material, including pharmaceuticals, which are products of a biological organism. Typical biological materials of interest can include drugs, organic oils, sebum, bacteria, epithelial cells, amino acids, proteins, DNA, and the like.

As used herein, “bonded” and “bonding,” when used in connection with nanodiamond contact with biological materials, refers to bonding such as covalent bonding, ionic bonding, mechanical bonding, van der Waals attractions, hydrogen bonding, or other intermolecular attractive forces.

Concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited.

### Theory

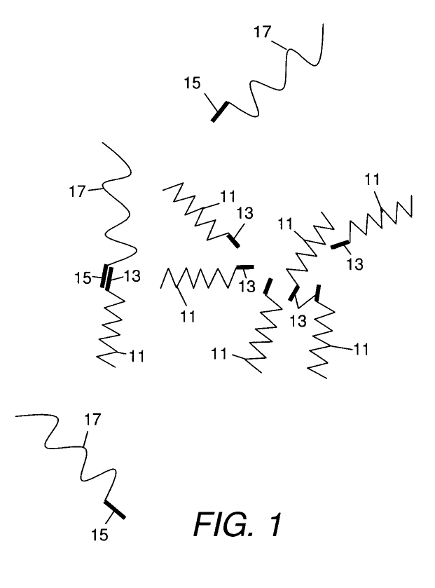

Nanodiamond powders due to very small particle size (2-10 nm) and with majority of carbon on the surface present a class of nanomaterials with tunable surface properties. FIG. 1 is a schematic illustration of how molecules react under normal prior art conditions.

As stated in the “Background of the Invention”, chemical functional groups, drug molecules 11, typically in solution, randomly orient themselves and by random chance align in the proper orientation to have an active chemical site 13 make contact with the proper active chemical site 15 of a molecule in another chemical entity 17. Chemical entity 17 here may be a pain receptor of a patient if drug molecules 11 are pain reducing molecules. Chemical entity 17 may be a cholesterol precursor if the drug molecules 11 are cholesterol reducing drug molecules. Similarly other drug molecules and their corresponding reactants may be used.

If these active sites 13 are hidden inside a clump of molecules 11 (shown in the center of the figure) or otherwise inaccessible, the chances that the active sites 13 make contact another active site 15 of the microbe is reduced. It is better if the active sites are exposed.

Since each of these are based upon the random motion of molecules in solution, the chances that an active site of a molecule having the proper orientation makes contact with an active site of the proper molecule is a matter of chance. The greater the number of molecules and active sites in solution, the greater the chances of the desired chemical bindings between active sites. Therefore, by exposing and holding the active sites of the drugs 11 outward in an exposed, fixed orientation and gradually varying the orientations across a surface, there will be an orderly array of exposed active sites.

Molecules flowing in a solution are randomly dispersed and oriented. Also, since drugs flow in solution to attach to active sites on the microbe, it is important to have a large amount of the drugs in solution, increasing the local concentration and the potential of attaching to an active site.

Also, the orderly arrangement of active sites must be able to move to meet up with the molecules of the microbe to interact with the active sites of these molecules. Therefore, this orderly arrangement must be mobile.

Foreign objects in the body are identified by the body's immune system and either destroyed or ejected from the body. The immune cells of the body may seek out and kill, or engulf and carry foreign objects out of the body. This would greatly reduce the efficacy of any drug introduced into the body which is recognized as a foreign substance.

The body ignores particles which are 10 Nanometers (nm.) or smaller. This may be due to the fact that there are many naturally occurring objects in the body fluids which are 10 nm. or smaller.

Nanodiamonds (“ND”) are diamonds which are 6 nm or smaller. These are typically produced according to the process explained in U.S. Pat. Nos. 5,916,955 and 5,861,349 assigned to NanoBlox, Inc. issued June and January 1999 respectively. In this process, carbon is converted in an explosive process to create NDs in which the vast majority of the NDs produced is approximately 6 nm.

The compositions of the present invention can include a plurality of nanodiamond and/or functionalized nanodiamond particles. Suitable nanodiamond particles can have an average size of from about 0.5 nm to about 50 nm. In some embodiments the plurality of nanodiamond particles can have an average size from 1 nm to about 10 nm, preferably from about 4 nm to about 8 nm, and most preferably about 5 nm. The concentration of nanodiamond particles will vary depending on the composition and the desired effect, as discussed in more detail below. As a practical matter, the plurality of nanodiamond particles is typically about 1 wt % to about 80 wt % of the composition. Nanodiamond particles can be formed using a number of known techniques such as shock wave synthesis, CVD, and the like. Currently preferred nanodiamond particles are produced by shock wave synthesis.

In addition to mechanical strength, introduction of nanodiamond particles to a biologically active composition can provide a number of beneficial properties. One of such beneficial properties is an impressive ability of nanodiamonds to absorb and become bound to other organic materials. Carbon atoms are very small (about 1.5 angstroms); thus, various forms of carbon can pack to form a high atomic concentration. In fact, diamond has the highest atomic concentration (176 atom/nm.sup.3) of all known materials. This high atomic concentration contributes to the exceptional hardness of diamond. As a result, any given surface area of a nanodiamond particle can include many more potential binding sites than other nanoparticles of the same size. This enables the maintenance of higher concentrations of biological active materials per unit area, thus yielding improved efficacy and/or lower dose rates.

Diamond is known to be non-toxic and biocompatible. Specifically, at temperatures below about 500 degree. C., diamond typically does not react with other materials. Further, diamond is compatible with most biological systems. As such, diamond is ideal for use in medical applications, e.g., artificial replacements (joint coatings, heart valves, etc.), and will not deteriorate over time.

Although diamond is highly stable, if the nanodiamond surface is free of adsorbent or absorbent, i.e. clean, it is thought that carbon atoms on the surface contain unpaired electrons that are highly reactive. As a result, nanodiamond particles can readily bond to and effectively absorb a variety of atomic species. For example, small atoms such as H, B, C, N, O, and F can be readily adsorbed on the nanodiamond surface, although other atoms can also be absorbed. Hence, nanodiamond particles, with their vast number of surface atoms, can hold a large amount of such adsorbed or covalently bound atoms. For example, nanodiamond particles are capable of absorbing almost as many hydrogen atoms as the number of carbon atoms. Thus, nanodiamond particles can be used as storage sites for hydrogen. In addition, those small atoms are building blocks, e.g., H, CO, OH, COOH, N, CN and NO, of organic materials including biological molecules. Consequently, nanodiamond particles can readily attach to amino acids, proteins, cells, DNA, RNA, and other biological materials, and nanodiamond particles can be used to remove skin oils, facial oils, compounds that result in body odor, bacteria, etc.

Further, nanodiamonds are typically smaller than most viruses (10 to 100 nm) and bacteria (10 to 100 μm). Therefore, nanodiamond can be used to penetrate the outer layers of viruses and bacteria and then attach to RNA, DNA or other groups within the organism to prevent the virus or bacteria from functioning. Similarly, nanodiamond can be used in conjunction with known drug delivery mechanisms to treat cancer or acquired immune deficiency syndrome.

In recent years, nanoparticles of diamond have become commercially available. Such nanodiamond particles are commonly formed by explosion. However, instead of graphite being compressed with a shock wave, the Composition B/Dynamite (e.g. TNT and RDX mixture) itself is converted to nanodiamond during less than a microsecond when both the pressure and temperature are high, i.e. over 20 GPa and 3000 degree. C. Nanodiamonds so formed are typically smaller than 10 nm (e.g. 5 nm) and tend to have a very narrow size distribution, i.e. from about 4 nm to about 10 nm. Moreover, the surface of these nanodiamonds contains diamond or diamond-like carbon, such as bucky balls (C60), layered shells, and amorphous carbon. Thus, these nanodiamonds are extremely hard without sharp corners.

As a result of the molecular structure and properties of nanodiamond, it possesses unique potential for surface modification and organic functionalization. Compositions and methods of using functionalized nanodiamond which improve desirable properties of various biological and medical compositions have been successfully prepared.

Nanomedicine, for example, focuses on applications of nanotechnology to achieve breakthroughs in healthcare. Nanomaterials are currently being investigated for improvements in drug delivery systems. Improvements in this area hold promise to lower drug toxicity, reduce treatment costs, and improve bioavailability of certain drugs. Nanotechnology has been used to improve injectable drugs, providing next generation drugs with improved dosage forms making administration of the drugs easier. In addition, nanostructured silicon materials have been designed to store active compounds which eventually become released in a time dependent manner as the silicon dissolves.

Combining concepts of nanotechnology, biotechnology and medicine, scientists are developing powerful tools to better understand the structure and function of organisms. Such understanding may ultimately develop better treatments options particularly at the molecular level and may translate into enhanced detection and treatment options. Use of nanoparticles in caner treatments is beginning to show promising results, allowing targeting of various drugs to specific sites and tumors, using lower dosages and ultimately reducing side effects. Current platforms for which nanoparticles are considered include, designing the nanoparticles to overcome physiological barriers, i.e. blood-brain barriers, manipulation of surfaces of the particles to avoid immunological detection, use as drug delivery, and tissue targeting.

Of particular promise for use in biomedical applications, including drug-delivery mechanisms, are nanodiamonds. Discovered in the 1960's, nanodiamonds are a unique nano-sized molecule yet to be fully understood or developed. Nanodiamonds are produced by detonation synthesis, a procedure which produces the approximate 5 nanometer carbon particles. Nanodiamonds feature a diamond core which is covered by graphite layers and amorphous carbon. Nanodiamonds are attractive for various commercial uses because of its diamond core and large surface containing many functional groups. Such functional groups can be manipulated making them an attractive potential tool in the biomedical field. Moreover, since the nanodiamonds are carbon-based, biocompatible and non-toxic, they can be used in developing novel drug delivery systems, drug diagnostics, and medical imaging.

A process for organic functionalization methods for small detonation diamond agglomerates was disclosed by Kruger et al. (see Surface functionalization of detonation diamond suitable for biological applications, J. Mater. Chem., 16, 2322-2328 (2006)). The process described in the reference differs from the process developed by the inventors of the instant invention. As disclosed by Kruger et al, the functionalization of the nanodiamond is performed by a salination process. A saline linker was added to the nanodiamond, allowing it the ability to be linked to small peptides. However, the nanodiamond used in these experiments contain multiple detonation surfaces resulting in non-homogenous surfaces. While such nanodiamonds may have —OH functional groups as a result of the process, other functional groups are attached to the surface as well. Existence of multiple types of various functional groups on the surface has the effect of changing the valiancy on the nanodiamond, affecting the overall biology of the attached peptides, and may be toxic to the body. In addition, the conditions used in the process are not stable in acidic conditions. Such acid instability results in limiting use of the nanodiamond such as synthesizing procedures in basic conditions. Moreover, acidic environments within the body may cause degradation of the nanodiamond making it ineffective for many biomedical uses.

These can be cleaned to take any graphite off of the surface to result in pure NDs of about 5 nm in diameter.

NDs have been shown to stabilize suspensions and solutions and greatly increase solubility of substances in solutions.

NDs have also been known to be functionalized to attach fluorine groups to its surface. This was intended to alter the surface composition of the NDs, but not for the purposes similar to that of the present invention.

Since particles of 10 nm or less are allowed to freely pass through a mammalian body, it is believed that these may be perfect transport vehicles for many different drugs. Therefore, drug molecules could be attached to the NDs to create a drug-ND complex.

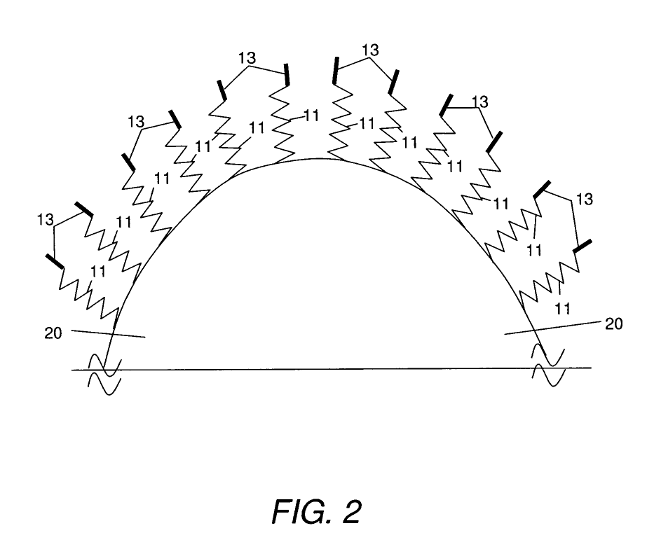

FIG. 2 is a schematic microscopic view of a portion of a nano-diamond showing the structure of chemical entities attached to the surface of the nano-diamond.

The ND 20, exhibits a spherical shape. Here one is covered with a plurality of drugs 11. The drugs 11 are fixed in an orientation which extends them outwardly.

This causes the active sites 13 of each of the drugs 11 to be exposed and point outwardly. Since the surface of ND 20 is curved, as one moves along the surface in any direction, the orientation of the drugs 11 and their active sites 13 changes slightly, allowing a continuum of orientations for the active sites 13. Therefore, there is a greater chance of the active sites of randomly oriented molecules to come in contact with the active sites 13 of drugs 11 having the proper orientation for reaction.

Therefore, if one were to supply an orderly arrangement of such drug molecules covering the surface of the NDs with the active sites facing outwardly, it is believed that the efficacy of the drugs would be greatly increased.

It was found, by extensive trial and error, that the efficacy of substances can be amplified by attachment to functionalized NDs. Modifying NDs has two major components. The first component is to cover the surface of the NDs with an intermediate compound. It was found that by replacing covalently attaching amine radicals to the exposed carbon chains of the NDs creates a platform which may then be used to attach other functional groups.

The second step would be to attach functional molecules and/or groups to the exposed amine groups.

### Covalent Functionalization of Nanodiamond

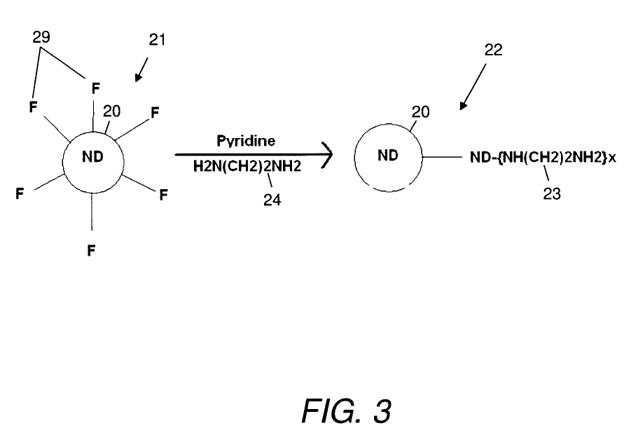

FIGS. 3, 4 and 5 illustrate three different processes creating nano-diamonds coated with an intermediary compound according to the present invention.

1. The nanodiamond (ND) is comprised of carbon chains which end with surface molecule. Surface preparation moieties, such as fluorine are attached to a plurality of the surface molecules to produce nanodiamond covered by fluorine atoms. This fluorinated-ND is a powder shown as entity 21 of FIG. 3.

Fluoro-ND powder is reacted with anhydrous ethylenediamine (H₂N(CH₂)₂NH₂) in the presence of pyridine (PY). This takes place at about 130 degree C. for 24 hours under a nitrogen atmosphere. The fluorine moieties on the ND surface will be eliminated by formation of HF molecules and will be replaced with the ethylenediamine.

This substance is filtered, washed and then dried in vacuum oven at 70 degree Centigrade overnight to produce the complex 22 of FIG. 3. Ethylenediamine is the intermediary 23 of complex 22 that may be replaced with desired drug molecules in subsequent processes.
2. In a different reaction process, the fluorinated-ND powder can be used to react with multiamino-organsilane (CH3O)3Si(CH2)3NHCH2CH2NH2) in the presence of HF to produce nanodiamond with amino-nanodiamond moieties. The list of multiamino organo silane such as AEA (N-2-amino-ethyl-3-aminopropyl-trimethoxysilan, trimethoxysilylpropyl-diethylenetrianamine (DETA), 3-aminopropyltriethoxysilane APTES are given here as an example can be used for this process.

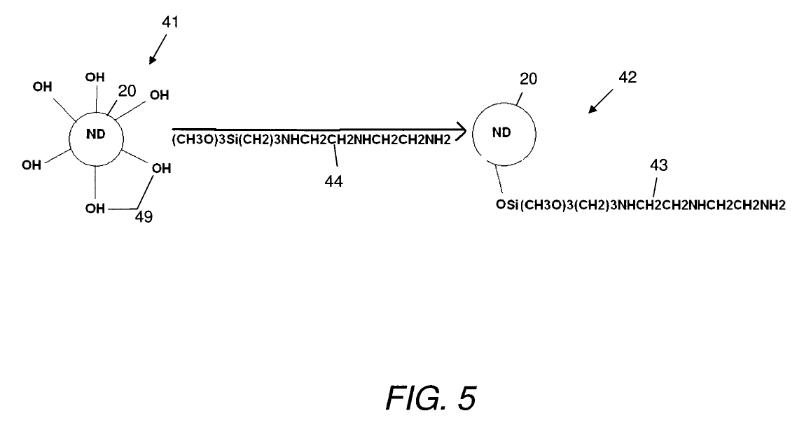

The reaction of DETA in the presence of HF and fluoro-ND 21 is shown in FIG. 3. The resulting complex 32 includes an intermediary 33 that is essentially DETA coating the surface.
3. In FIG. 5, ND is prepared with Hydroxyl surface moieties to create the complex 41.

Complex 41 can react with multiamino-organosilan groups 44 such as AEA (N-2-amino-ethyl-3-aminopropyl-trimethoxysilan, trimethoxysilylpropyl-diethylenetrianamine (DETA), 3-aminopropyltriethoxysilane APTES. This reaction will provide Amino-nanodiamond terminal moieties as intermediaries 43 covering the surface of ND 20.

Currently pending U.S. Patent Application “Functionalization of Nanodiamond Powder Through Fluorination and Subsequent Derivatization Reactions” by Khabashesku et al, Ser. No. 10/996,869 filed Nov. 24, 2004, owned by Rice University, Houston, Tex. describes two methods of coating nanodiamonds with intermediary moieties similar to methods 1 and 2 above. These methods may also be used to attach intermediary moieties to coat the nanodiamond surface.

### Step 2—Functionalization

The next step would be to replace the intermediaries 23, 33, 43 of FIGS. 3, 4, 5, respectively coating the nanodiamond surface, and attach functional molecules and/or groups.

Covalent Functionalization of Nanodiamond with a Drug.

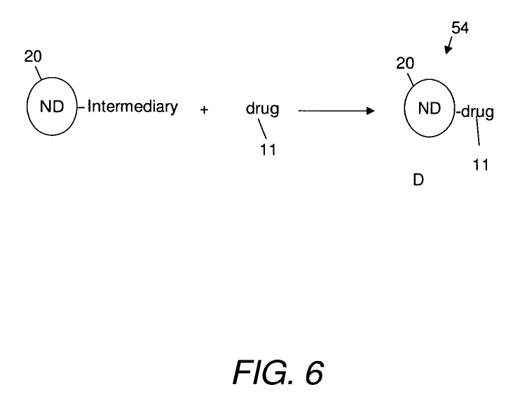

FIG. 6 is an illustration of the entities of the second part of chemical reaction. The intermediaries 23, 33, 43 are then replaced by the desired drug 11. This results in the drug 11 coating ND 20 according to the present invention.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

### Pain Reducing Drugs

A drug 11 attached to the aminated NDs could be an analgesic. This may fall under the categories of:

### 1. Opium and Alkaloids:

codeine, morphine, opium, laudanum and paregoric;

### 2. Semi Synthetic Opium Derivatives Including:

Acetyldihydrocodone, Benzylmophine, Desomorphine, Dihydrocodone, Dihydromorphine, Ethylmorphine, Diamorphine, Hydrocodone, Hydromorphinol, Hydromorphone, Nicocodeine, Nicodicodeine, Nicomorphine, Oxycodone, Oxymorphone, Thebacon

3. Synthetic Opiuds including:

Alohaprodine, Anileridine, Buprenorphine, Butorphanol,

Dextromoramine, Dextropropoxyphene, Dezocine, Fentanyl, Ketobemidone, Levorphanol, Methadone, Meptazinol, Nalbuphine, Pentazocine, Propoxyphene, Propiram, Pethidine, Phenazocine, Piminodine, Piritramide, Tapentadone, Tilidine, Tramadol

### 4. Pyrazolones Including:

Ampyrone/Aminophenazone, Metamizole, Phenazone

### 5. Cannabinoids Including:

Ajulemic acid, AM404, Cannabidiol, Cannabis, Nabilone, Tetrahydrocannabinol

### 6. Aniledes Including:

Paracetamol (acetaminophen), Phenacetin, Propacetamol

### 7. Propionic Acid Class Including:

Fenoprofen, Flurbiprofen, Ibuprofen, Ketoprofen, Ketoprofen, Naproxen, Oxaprozin

### 8. Oxicam Class Including:

Meloxicam, Piroxicam

### 9. Acetic Acid Class Including:

Diclofenac, Indometacin, Ketorolac, Nabumetone, Sulindac, Tolmetin

### 10. Non-steroidal Anti-Inflammatories, COX-2 Inhibitors Including:

Celecoxib, Rofecoxib, Valdecoxib, Parecoxib, Lumiracoxib.

### 11. Non-steroidal Anti-Inflammatories, Anthranilic Acid (Fenamate) Class, Including:

Meclofenamate, Mefenamic acid

### 12. Non-steroidal Anti-Inflammatories, Salicylates Including:

Aspirin (Acetylsalicyclic acid), Benorylate, Diflunisal, Ethenzamide, Magnesium salicylates, Salicin, Salicylmide, Salsalate, trisalate

### Cholesterol Reducing Agents/Drugs

Cholesterol is the major, and probably the sole precursor of bile acids. During normal digestion, bile acids are secreted via the bile from the liver and gall bladder into the intestines. Bile acids emulsify the fat and lipid materials present in food, thus facilitating absorption. A major portion of the bile acids secreted is reabsorbed from the intestines and returned via the portal circulation to the liver, thus completing the enterohepatic cycle. Only very small amounts of bile acids are found in normal blood serum.

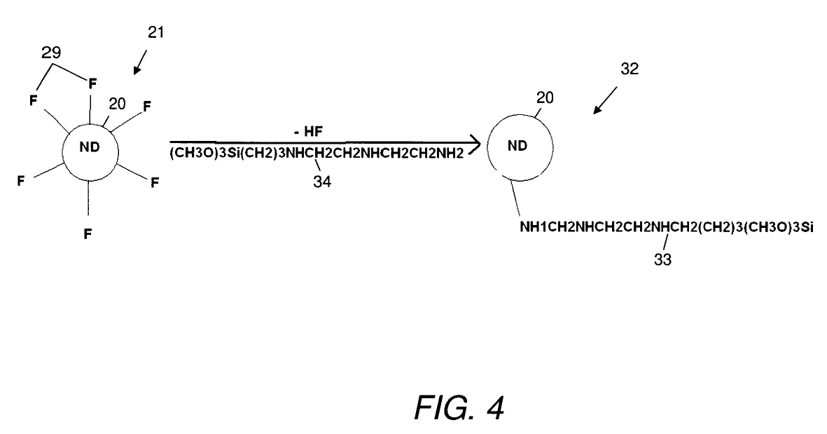

It is believed that aminated ND, entity “C” shown in FIG. 4 and described above, binds bile acids in the intestine forming a complex that is excreted in the feces. This nonsystemic action results in a partial removal of the bile acids from the enterohepatic circulation, preventing their reabsorption. Since aminated ND is an anion exchange resin, the chloride anions of the resin can be replaced by other anions, usually those with a greater affinity for the resin than the chloride ion.

Aminated ND is hydrophilic, but it is virtually water insoluble (99.75%) and it is not hydrolyzed by digestive enzymes. The high molecular weight polymer in aminated ND apparently is not absorbed.

The increased fecal loss of bile acids due to aminated ND administration is believed to lead to an increased oxidation of cholesterol to bile acids. This results in an increase in the number of low-density lipoprotein (LDL) receptors, increased hepatic uptake of LDL and a decrease in beta lipoprotein or LDL serum levels, and a decrease in serum cholesterol levels. Although aminated ND produces an increase in the hepatic synthesis of cholesterol in man, serum cholesterol levels fall.

It is believed that this fall in serum cholesterol is secondary to an increased rate of clearance of cholesterol-rich lipoproteins (beta or low-density lipoproteins) from the blood plasma. Serum triglyceride levels may increase or remain unchanged.

Alternatively, a cholesterol-reducing drug, such as Cholestid® may be attached to the aminated nanodiamond as described above for analgesic drugs 11 of FIG. 4.

### Method of Delivery

There are various known methods of introducing the ND-drug complexes into the body of the patient. For example, the most obvious would be in a pill or liquid form which the subject ingests. This is only allowable for drugs which are not effected by the acids of the digestive tract.

The ND-drug complexes may injected, administered by air gun, nose spray, be inhaled, or used as a suppository.

The ND-drug complexes may be used as a disinfectant as an air spray, applied to the hands, or incorporated into materials around the patient, such as sheets and bedding.

They may also be incorporated into medical disposables, such as surgical drapes, bandages and disposable coverings.

Even though this description was performed for a pain reduction and cholesterol reducing drugs, it is believed that this applies to increasing the efficacy of other drugs and preparations. If these other drugs are used instead of the listed drugs and attached to the surface of NDs, their efficacy will also increase.

Even though this invention was described in terms of nanodiamonds, nanocarbon particles may also be used with this invention.

Since other modifications and changes varied to fit particular operating requirements and environments will be apparent to those skilled in the art, the invention is not considered limited to the example chosen for the purposes of disclosure, and covers all changes and modifications which do not constitute departures from the true spirit and scope of this invention.

What is claimed is:

1. A method of enhancing efficacy of a drug 11 having an active site, comprising the steps of:
a) acquiring a plurality of nanodiamond (ND) particles 20 having a plurality of carbon chain surface molecules on its surface, the ND particles 20 having a diameter of less than 10 nanometers;
b) attaching surface preparation moieties 29, 49 to a plurality of said surface molecules to prepare said surface for further reactions;
c) covalently attaching a plurality of intermediate entities 24, 34, 44 to the surface molecules of the ND particles by replacing the surface preparation moieties 29, 49; and
c) replacing at least a portion of the intermediate entities 24, 34, 44 attached to the surface molecules of the ND particles 20 with said drug molecules 11 to create functionalized ND particles with increased efficacy.

2. The method of claim 1, wherein, the step of replacing comprises:
replacing at least a portion of the intermediate entities 24, 34, 44 attached to the surface molecules of the ND particles 20 with said drug molecules 11 such that a plurality of active sites of said drug molecules 11 point away from the ND particle 20 exposing them for enhanced activity and enhanced drug efficacy.

3. The method of claim 2, wherein the drug molecules 11 are selected from the group consisting of:
analgesics, blood pressure reducers, beta-blockers and cholesterol reducing drugs.

4. The method of claim 3, wherein the analgesics are selected from the group consisting of:
opiates, alkaloids, semi synthetic opium derivatives, synthetic opiuds, Pyrazolones, Cannabinoids, Aniledes, Propionic Acid Class, Oxicam class, Acetic acid class, Non-steroidal anti-inflammatories, COX-2 inhibitors, Non-steroidal anti-inflammatories, Anthranilic acid (fenamate) class, Non-steroidal anti-inflammatories and Salicylates.

5. The method of claim 1 wherein the surface preparation moieties 29, 49 are selected from the groups consisting of: fluorine and hydroxyl groups.

6. A method of enhancing the efficacy of drug molecules comprising the steps of:
a) acquiring nanodiamond (ND) particles having carbon chain surface molecules created by a detonation process with the majority of the particles having a diameter of less than 10 nm;
b) processing the surface of the ND by attaching hydroxile groups 49 to a plurality of said surface molecules of ND 20;
c) replacing the hydroxyl groups 49 with intermediary groups 43;
f) replacing the intermediate groups 43 with said drug molecules 11 to result in functionalized ND particles 54 exhibiting enhanced efficacy when compared to prior art drugs.

7. The method of claim 6, wherein, the step of replacing the intermediate groups 43, comprises the steps of:
replacing at least a portion of the intermediate entities 24, 34, 44 attached to the surface molecules of the ND particles 20 with said drug molecules 11 such that a plurality of active sites of said drug molecules 11 point away from the ND particle 20 exposing them for enhanced activity and enhanced drug efficacy.

8. The method of claim 6, wherein the drug molecules 11 are selected from the group consisting of:
analgesics, blood pressure reducers, beta-blockers and cholesterol reducing drugs.

9. The method of claim 6, wherein the analgesics are selected from the group consisting of:
Opiates, alkaloids, semi synthetic opium derivatives, synthetic opiuds, Pyrazolones, Cannabinoids, Aniledes, Propionic Acid Class, Oxicam class, Acetic acid class, Non-steroidal anti-inflammatories, COX-2 inhibitors, Non-steroidal anti-inflammatories, Anthranilic acid (fenamate) class, Non-steroidal anti-inflammatories and Salicylates.

10. The method of claim 6 wherein the surface preparation moieties 29, 49 are selected from the groups consisting of: fluorine and hydroxyl groups.

11. A method of enhancing the efficacy of drug molecules 11 comprising the steps of:
a) acquiring nanodiamond (ND) particles 20 having carbon chain surface molecules created by a detonation process with the majority of the particles having a diameter of less than 10 nm;
b) processing the surface of the ND 20 by attaching hydroxile groups 49 to a plurality of said surface molecules of ND 20;
c) replacing the hydroxyl groups 49 with intermediary groups 43;
f) replacing the intermediate groups 43 with said drug molecules 11 to result in functionalized ND particles 54 exhibiting enhanced efficacy when compared to prior art drugs.

12. The method of claim 5, further comprising the step of:
administering the functionalized ND particles 54 to a patient by injection.

13. The method of claim 5, further comprising the step of:
administering the functionalized ND particles 54 to a patient by compressed air gun.

14. The method of claim 5, further comprising the step of:
administering the functionalized ND particles 54 to a patient as a nose spray.

15. A method of enhancing the solubility of drug molecules 11 comprising the steps of:
a) acquiring nanodiamond (ND) particles 20 having carbon chain surface molecules created by a detonation process with the majority of the particles having a diameter of less than 10 nm;
b) processing the surface of the ND 20 by attaching hydroxile groups 49 to a plurality of said surface molecules of ND 20;
c) replacing the hydroxyl groups 49 with intermediary groups 43;
f) replacing the intermediate groups 43 with said drug molecules 11 to result in functionalized ND particles 54 exhibiting enhanced solubility when compared to prior art drugs.
